(19) United States
(12) Patent Application Publication
(10) Pub. No.: US 2020/0060652 A1
(43) Pub. Date: Feb. 27, 2020

(54) NEURAL NETWORKS-ASSISTED CONTRAST ULTRASOUND IMAGING

(71) Applicant: The Board of Trustees of the Leland Stanford Junior University, Stanford, CA (US)

(72) Inventors: Jeremy J. Dahl, Palo Alto, CA (US); Dongwoon Hyun, Sunnyvale, CA (US); Leandra L. Brickson, Palo Alto, CA (US)

(21) Appl. No.: 16/539,596

(22) Filed: Aug. 13, 2019

Related U.S. Application Data
(60) Provisional application No. 62/721,950, filed on Aug. 23, 2018.

(51) Int. Cl. A61B 8/08; A61B 8/14; G06N 3/08

(52) U.S. Cl. CPC A61B 8/481; G06N 3/08; A61B 8/5269; A61B 8/14

(57) ABSTRACT

A method of nondestructively detecting targeted contrast agents in real-time is provided that includes using a neural network (NN) beamformer, where an input of the NN includes ultrasound transducer channel data from a dual-frequency pulse-echo acquisition from a medium that may contain targeted contrast agents, where an output of the NN is an image of pixel-wise probability of the targeted contrast agent presence, where the NN nondestructively distinguishes the targeted contrast agent from tissue and noise by exploiting characteristic differences in responses of the targeted contrast agent versus responses from the tissue and noise present in the channel data of the dual-frequencies, where the NN is trained to operate according to destructive-subtraction ultrasound molecular imaging datasets that are used as a ground truth.

### CROSS-REFERENCE TO RELATED APPLICATIONS

This application claims priority from U.S. Provisional Patent Application 62/721,950 filed Aug. 23, 2018, which is incorporated herein by reference.

### STATEMENT OF GOVERNMENT SPONSORED SUPPORT

This invention was made with Government support under contract EB022770 awarded by the National Institutes of Health. The Government has certain rights in the invention.

### FIELD OF THE INVENTION

The current invention relates to microbubble detection. More specifically, the invention relates to a method of detecting targeted microbubbles nondestructively using an deep neural network beamformer that processes channel data from dual-frequency transmissions.

### BACKGROUND OF THE INVENTION

Ultrasound imaging is attractive as a medical imaging modality because it is low cost, portable, non-invasive, and does not utilize ionizing radiation. However, conventional ultrasound imaging lacks the molecular specificity of alternative modalities such as magnetic resonance imaging and positron emission tomography. Recently, ultrasound molecular imaging (USMI) has been enabled by the introduction of targeted microbubbles (MBs). MBs are micron-sized gas bubbles encapsulated in a lipid shell, and are commonly used as an ultrasound contrast agent because of their strong scattering properties. The shells of MBs can be conjugated to bind to desired biomarkers with high specificity, and the bound MBs are subsequently detected using ultrasound. Thus, USMI can be used to detect molecular biomarkers with high specificity and high sensitivity.

USMI enables a wide range of applications, including the early detection of cancer. For instance, a biomarker associated with the development of tumor neovasculature called VEGFR-2 has been successfully targeted using MB contrast agents in preclinical studies for the detection of breast, prostate, and ovarian cancers in animal models.

However, clinical translation of USMI to human imaging faces several unique challenges that are often circumvented in preclinical imaging. For instance, preclinical tumors are often more accessible than human tumors (e.g., subcutaneous vs. deep). Most significantly, preclinical imaging studies commonly employ destructive-subtraction imaging (see FIG. 1), where destructive pulses are used to burst the MBs, and images acquired post-burst are subtracted from pre-burst images, leaving behind only MB signals. Destructive pulses are necessary to visualize the MBs because current state-of-the-art beamforming techniques provide insufficient suppression of tissue background and noise. However, bursting of the MBs can lead to significant damage of the vasculature and surrounding tissue, and may have additional bioeffects that are yet undiscovered. In a first-in-human study of USMI, destructive pulses were not used due to patient safety concerns, leading to poor tissue background suppression.

Moreover, destructive pulses intrinsically cannot be used for real-time imaging. Each time the MBs are destroyed, they must be replenished and given time to bind to the biomarkers (often upwards of 10 min.), leading to long examination times and potentially requiring higher dosages.

What is needed is a method of using USMI detect bound MBs nondestructively, allowing the clinician to freely interrogate the tissue for MBs in real time until they can arrive at a diagnosis.

### SUMMARY OF THE INVENTION

To address the needs in the art, a method of nondestructively detecting targeted contrast agents in real-time is provided that includes using a neural network (NN) beamformer, where an input of the NN includes ultrasound transducer channel data from a dual-frequency pulse-echo acquisition from a medium that may contain targeted contrast agents, where an output of the NN is an image of pixel-wise probability of the targeted contrast agent presence, where the NN nondestructively distinguishes the targeted contrast agent from tissue and noise by exploiting characteristic differences in responses of the targeted contrast agent versus responses from the tissue and noise present in the channel data of the dual-frequencies, where the NN is trained to operate according to destructive-subtraction ultrasound molecular imaging datasets that are used as a ground truth.

According to one aspect of the invention, the NN is configured to accept interleaved fundamental and harmonic frequency channel data, where the fundamental frequency acquisition includes one set of pulses at the imaging frequency, where the harmonic frequency acquisition includes two sets of pulses at half of the imaging frequency with opposite polarities that are summed.

In another aspect of the invention, the NN is configured to accept fundamental and harmonic frequency channel data, where the fundamental frequency acquisition includes one set of pulses at half of the imaging frequency, where the harmonic frequency acquisition includes a sum of said set of pulses at half of the imaging frequency with a second set of pulses at half of the imaging frequency with opposite polarities.

In a further aspect of the invention, the dual-frequency pulse-echo acquisitions are performed using a plane wave or diverging wave synthetic transmit aperture technique.

In one aspect of the invention, the channel data acquisition includes the radiofrequency data acquired on all transducer elements.

According to another aspect of the invention, the channel data acquisition includes a downsampled form of the radiofrequency data acquired on all transducer elements.

In yet another aspect of the invention, the NN is trained to identify the contrast agents according to destructive-subtraction images that are used as the ground truth, where each destructive-subtraction image is formed by acquiring a pre-destruction image, eliminating the contrast agents from an imaging field of view using destruction, and subtracting a post-destruction image from the pre-destruction image, where the pre-destruction and post-destruction images are each formed using the best available temporal filtering techniques and beamforming methods.

In a further aspect of the invention, the pre-destruction and post-destruction images are reconstructed by using temporal filtering techniques that can include averaging a group of the channel data acquisitions comprising up to 30 frames and subsequently beamforming.

In a further aspect of the invention, the pre-destruction and post-destruction images are reconstructed using a beamforming method that can include delay-and-sum beamforming, or SLSC beamforming, where the destructive-subtraction images are further enhanced using manual segmentation and image post-processing to eliminate artifacts.

In yet another aspect of the invention, training of the NN includes obtaining a pre-destruction dual-frequency channel data acquisition, passing the dual-frequency channel data acquisition into the NN to estimate a map of pixel-wise probability of the presence of the contrast agent (ŷ), applying a strong destructive pulse to eliminate contrast agents from an imaging field of view and forming a ground truth destructive-subtraction image (y), and comparing the (ŷ) versus (y) using a loss function, and to update the parameters of the neural network to minimize the loss function during the training.

### BRIEF DESCRIPTION OF THE DRAWINGS

FIG. 1 shows destruction-subtraction imaging, where images are acquired before and after a strong destructive pulse. The post-burst image is subtracted from the pre-burst image, removing background signals and isolating the burst MBs, according to the current invention.

FIGS. 2A-2B show the neural network training and evaluation procedure that includes Estimating ŷ: The channel data for a fundamental and harmonic acquisition are acquired, downsampled, and passed into a fully convolutional neural network, which produces an estimate ŷ∈[0, 1]^{M×N}. Obtaining y: A strong destructive pulse is used to destroy the MBs and an additional harmonic dataset is acquired. In this example, a post-burst SLSC image is subtracted from pre-burst and is manually segmented to obtain a binary mask of ground truth y∈{0, 1}^{M×N}. Evaluation: A loss function L(ŷ, y) is used to compare ŷ and y, and during training, to update the parameters of the neural network, according to the current invention.

FIGS. 3A-3C show three example results from the test. Each group of 4 images shows the fundamental B-mode, harmonic B-mode, destruction-subtraction with manual segmentation (y), and nondestructive neural network output (ŷ). In the (3A) positive and (3B) negative controls, the neural network predicted the presence and absence of MBs as expected. In the (3C) mouse tumor, the network prediction is comparable to the destruction-subtraction-segmentation image, according to the current invention.

FIG. 4 shows the receiver operating characteristics (ROC) curve for the neural network detector in a mouse tumor with targeted MBs (FIG. 3C). The pixel-wise probability output of the network was thresholded into a binary mask, with the threshold swept from p=0 to p=1. The area under the ROC curve (AUC) was reported to be 0.90, according to the current invention.

FIG. 5 shows the soft Dice coefficients achieved as a function of learning rate by 9 different configurations of input data. (top row) No learning occurred when using only one set of fundamental frequency pulses (X_{f}: 10 MHz, Xₚ: 5 MHz) as input. Learning occurred when using the harmonic image alone as input (Xₕ: sum of sets of 5 MHz pulse of opposite polarity). (middle row) When providing Xₚ and Xₕ together as input to the network, learning occurred whether using channel data (Xₚₕ), the channel sum (Xₚₕ^{sum}), or the envelope detected image (Xₚₕ^{env}) as input. Learning was most consistently successful in a narrow range of learning rates when using channel data. (bottom row) When providing X_{f} and Xₕ together as input to the network, learning occurred and was consistently successful in a narrow range of learning rates when using channel data (X_{fh}), the channel sum (X_{fh}^{sum}), or the envelope detected images (X_{fh}^{sum}) as input. The highest Dice coefficients were achieved consistently when using the X_{fh} as input.

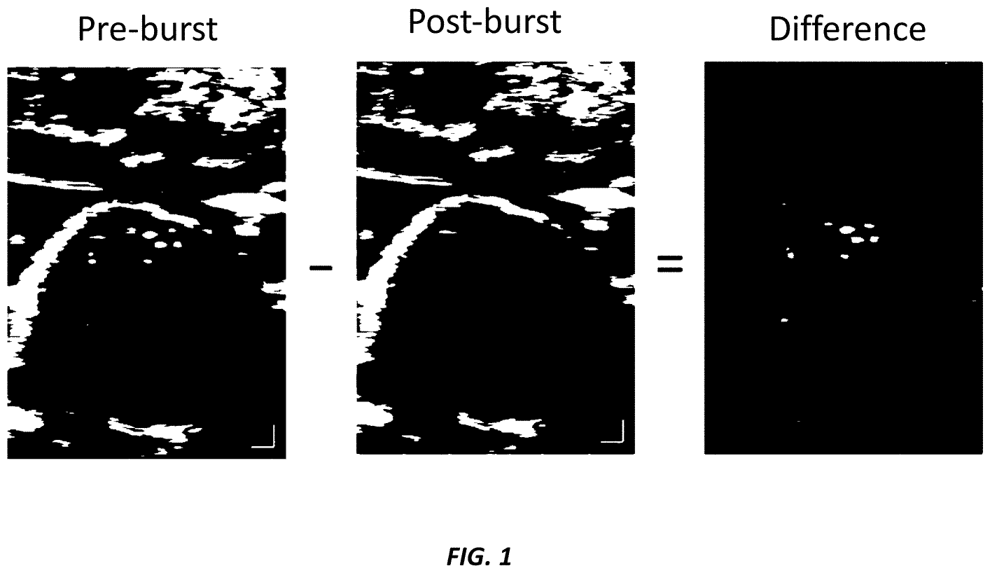

### DETAILED DESCRIPTION

Targeted microbubbles (MBs) enable ultrasound molecular imaging (USMI) by binding to specific biomarkers and producing strong reflections to ultrasound. However, current USMI techniques are not easily translatable for clinical use. In particular, preclinical studies often utilize destruction-subtraction imaging, wherein a strong destructive pulse is used to destroy MBs to confirm their locations. This approach is potentially unsafe, and is intrinsically not real-time. The current invention provides a method of nondestructively detecting targeted contrast agents in real-time that includes using a neural network (NN) beamformer. Here, an input of the NN includes ultrasound transducer channel data from a dual-frequency pulse-echo acquisition from a medium that may contain targeted contrast agents, where an output of the NN is an image of pixel-wise probability of the targeted contrast agent presence. The NN nondestructively distinguishes the targeted contrast agent from tissue and noise by exploiting characteristic differences in responses of the targeted contrast agent versus responses from the tissue and noise present in the channel data of the dual-frequencies. Finally, the NN is trained to operate according to destructive-subtraction ultrasound molecular imaging datasets that are used as a ground truth.

In one exemplary embodiment, the network is trained using a total of 20 USMI datasets acquired in a mouse model of hepatocellular carcinoma and in microvessel flow phantoms. The network was then evaluated on 5 distinct datasets: a positive control, a negative control, and three previously unseen mouse tumors. Across the 5 datasets, the neural network achieved a mean AUC of 0.91 and DC of 0.56 compared to the destruction-subtraction images. These results demonstrate that a neural network can nondestructively distinguish MBs from background tissue and noise by exploiting characteristic differences in their fundamental and harmonic responses. The nondestructive dual-frequency DNN beamformer enables safe and real-time USMI and can aid in the translation to clinical applications.

In another exemplary embodiment, networks were trained over a range of training hyperparameters using different combinations of input data configurations to identify the components essential to consistent and reproducible training. The networks did not train successfully when using fundamental frequency data alone and trained most successfully and consistently when using dual-frequency data as input.

The current invention advances a coherence-based beamforming technique for USMI, which utilized correlations among the transducer element signals to enhance MBs and suppress background tissue, further improving destruction-subtraction imaging. This previous technique showed that the channel data contain valuable information that is inaccessible via traditional delay-and-sum techniques. The current invention provides a clinically translatable method for forming high-quality USMI images nondestructively using a novel neural network beamformer.

In one aspect of the invention, the pre-destruction and post-destruction images are reconstructed using a beamforming method that can include delay-and-sum beamforming, or SLSC beamforming, or any other useful beamforming method, where the destructive-subtraction images are further enhanced using manual segmentation and image post-processing to eliminate artifacts.

According to one aspect of the invention, the NN is configured to accept interleaved fundamental and harmonic frequency channel data, where the fundamental frequency acquisition includes one set of 10 MHz pulses, where the harmonic frequency acquisition includes two sets of 5 MHz pulses with opposite polarities that are summed. Further, the NN is configured to accept fundamental and harmonic frequency channel data, where the fundamental frequency acquisition includes two sets of 5 MHz pulses with opposite polarities, where the harmonic frequency acquisition includes a sum of the two sets of 5 MHz pulses with opposite polarities.

In a further exemplary embodiment of the invention, USMI was performed in a mouse model of hepatocellular carcinoma in xenografted subcutaneous tumors. VEGFR-2-targeted BR55 MBs (Bracco, Milan, Italy) were injected via the tail vein. The MBs were allowed to circulate for 7 min. prior to imaging to provide sufficient time for targeted MBs to bind and for free MBs to be cleared. Low-mechanical-index nonlinear pulse sequences were used to perform USMI. The dual-frequency pulse-echo acquisitions are performed using a plane wave synthetic transmit aperture technique. Focal hotspots and inertial cavitation of the MBs were avoided by performing retrospective transmit beamforming of 7 plane waves transmitted at angles ranging from −9° to +9°. An L12-3v transducer was used to transmit pairs of 5 MHz pulses with inverted polarity and to receive signals bandpass filtered at 10 MHz. A Verasonics Vantage 256 research scanner and a custom GPU-based software beamformer were used to obtain radiofrequency (RF) signals from 128 transducer elements. The signals were demodulated and focused (i.e., delayed but not summed) into a M×N grid, yielding an IQ dataset of size C^{M×N×128}. A pixel spacing of 3 pixels per wavelength was used. In one aspect of the invention, the channel data acquisition includes a downsampled form of the radiofrequency data acquired on all transducer elements.

In one embodiment of the invention, the NN is trained to identify the contrast agents according to destructive-subtraction images that are used as the ground truth, where each destructive-subtraction image is formed by acquiring a pre-destruction image, eliminating the contrast agents from an imaging field of view using destruction, and subtracting a post-destruction image from the pre-destruction image, where the pre-destruction and post-destruction images are each formed by averaging a group of the channel data acquisitions comprising up to 30 frames and subsequently beamforming.

In a further example, receive USMI beamforming was performed using the coherence-based short-lag spatial coherence (SLSC) technique, which measured the average correlation coefficient across channel pairs with a spacing of at most 4 elements. Destruction-subtraction images were formed by acquiring images seven minutes after MB injection (pre-burst) and again after a strong destructive pulse (post-burst) and subtracting the post-burst SLSC image from the pre-burst SLSC image. These images were further manually segmented into a binary mask to eliminate obvious artifacts, resulting in a “ground truth” image denoted as y∈{0, 1}^{M×N}.

In the method of the current invention, a fully convolutional neural network is used to perform USMI. The network replaced the SLSC and destructive-subtraction components of beamforming. In one exemplary embodiment, a network was designed to accept the focused data demodulated at 10 MHz from two nondestructive pulse sequences: two 5 MHz inverted pulses (for second harmonic imaging) as well as a 10 MHz transmission (for fundamental imaging). Due to computational constraints, the focused channel data for each acquisition was downsampled to 16 channels via non-overlapping subaperture beamforming with subapertures of 8 elements each. Here, the acquired channel data from the nondestructive fundamental and harmonic acquisitions are denoted as X_{f} and Xₕ, respectively, and their concatenation is denoted X_{fh}. The output of the neural network is the pixel-wise probability of MB presence, ŷ∈[0, 1]^{M×N}. The neural network includes 4 repeated blocks of the Conv2D, BatchNorm, and ReLU layers, followed by a softmax operation to obtain the pixel-wise probability distribution. The network was implemented using TensorFlow.

In yet another aspect of the invention, training of the NN includes obtaining a pre-destruction dual-frequency channel data acquisition, passing the dual-frequency channel data acquisition into the NN to estimate a map of pixel-wise probability of the presence of the contrast agent (ŷ), applying a strong destructive pulse to eliminate contrast agents from an imaging field of view and forming a ground truth destructive-subtraction image (y), and comparing the (ŷ) versus (y) using a loss function, and to update the parameters of the neural network to minimize the loss function during the training.

More specifically, the network can be denoted as f_{θ}(X_{fh})=ŷ, where θ contains the learnable parameters. The parameters were updated via gradient descent by iterating over a training set (described below) so as to minimize a loss function L:

$${θ}^{*} = {arg min}_{θ} L ( {f}_{θ} ( {X}_{fh} ) , y ) \qquad ( 1 )$$

where a mixture of the cross-entropy loss function and soft Dice similarity coefficient was used:

$$\begin{matrix}ℒ ( \hat{y} , y ) = α {ℒ}_{XEnt} ( \hat{y} , y ) + ( 1 - α ) {ℒ}_{Dice} ( \hat{y} , y ) & ( 2 ) \\ {ℒ}_{XEnt} ( \hat{y} , y ) = - {\sum }_{p}^{MN} {y}_{p} log {\hat{y}}_{p} + ( 1 - {y}_{p} ) log ( 1 - {\hat{y}}_{p} ) & ( 3 ) \\ {ℒ}_{Dice} ( \hat{y} , y ) = 1 - \frac{{\sum }_{p}^{MN} 2 {\hat{y}}_{p} {y}_{p} + ϵ}{{\sum }_{p}^{MN} {\hat{y}}_{p} + {y}_{p} + ϵ} , & ( 4 )\end{matrix}$$

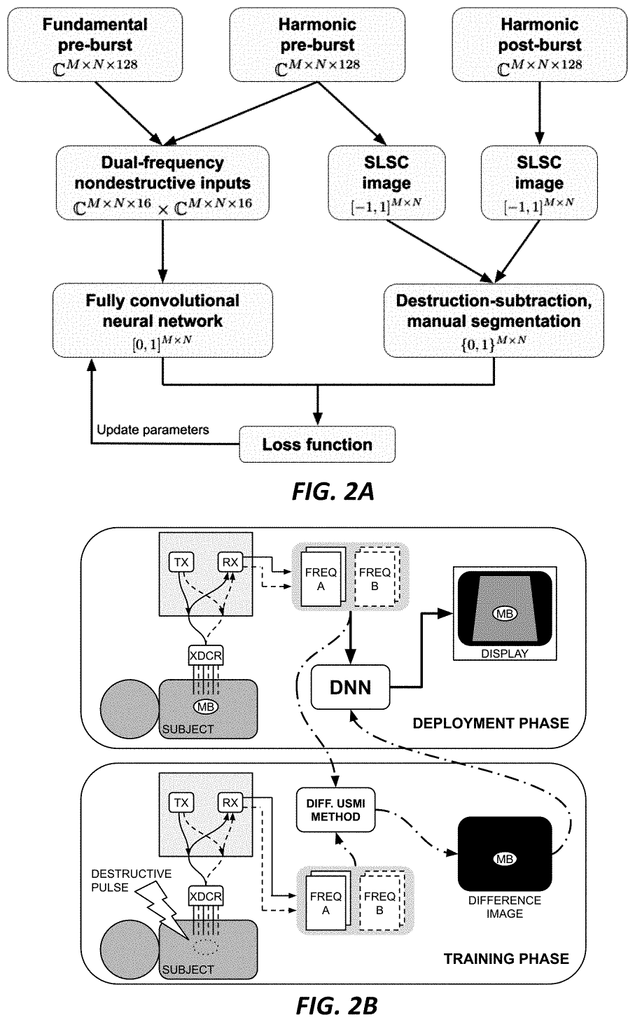

with p iterating over all M×N pixels, where α=0.3 was selected heuristically and ε=10⁻¹⁰ was used for numerical stability. The network was trained to minimize L for 125 epochs, i.e., iterations over the training dataset. FIGS. 2A-2B summarize the process for training and evaluating the neural network.

Regarding datasets and metrics, in one exemplary embodiment, a total of 25 distinct dual-frequency and destruction-subtraction datasets were obtained, with 5 acquisitions in a tissue-mimicking microvessel phantom (positive controls), one acquisition in a mouse abdomen prior to MB injection (negative control), and 19 acquisitions in mouse tumors 7 min. post-injection of targeted MBs. The 25 acquisitions were split into a training set of 20 and testing set of 5 acquisitions. Care was taken to ensure that the 25 acquisitions were acquired in different locations and tumors to avoid the inadvertent re-use of highly correlated data in the training and testing sets. For each acquisition, two frames of data were selected randomly to get two realizations of thermal noise. The datasets were then augmented two-fold by a left-to-right flip in both the azimuth and channel dimensions, and another two-fold by applying a constant π/3 radian complex phase rotation over the entire dataset, yielding a total of 160 training samples and 40 validation samples per input configuration. The network performance was then measured in the test dataset using the Dice coefficient and area under the ROC curve (AUC) metric.

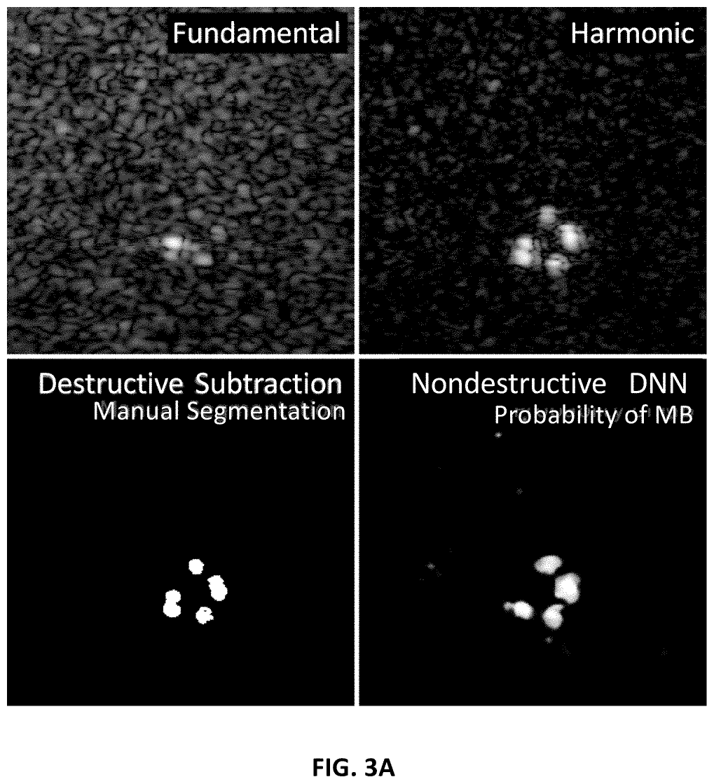

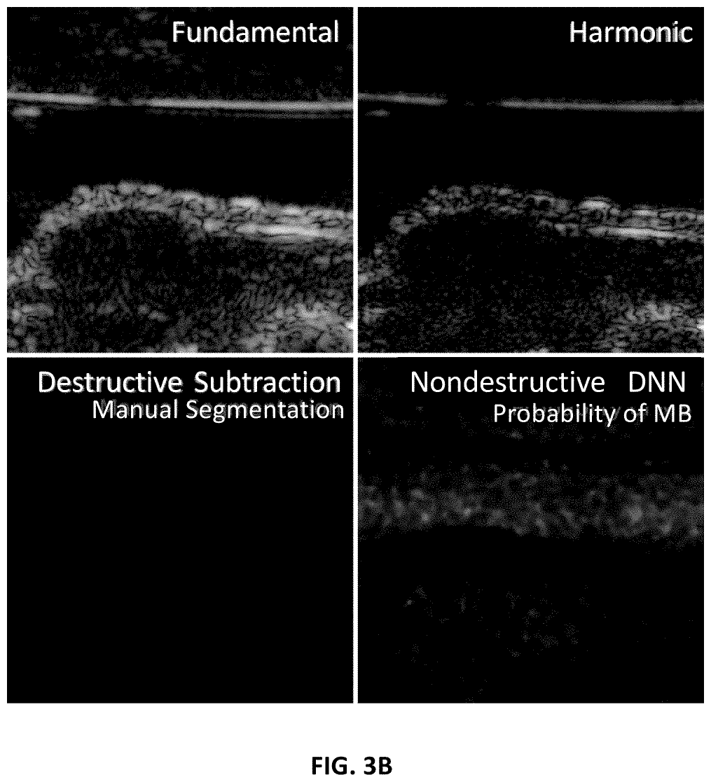

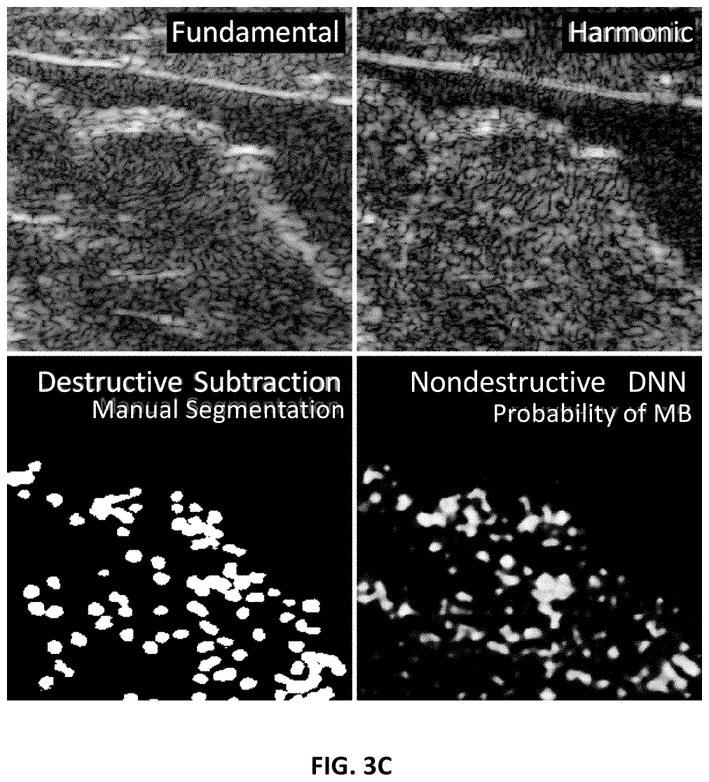

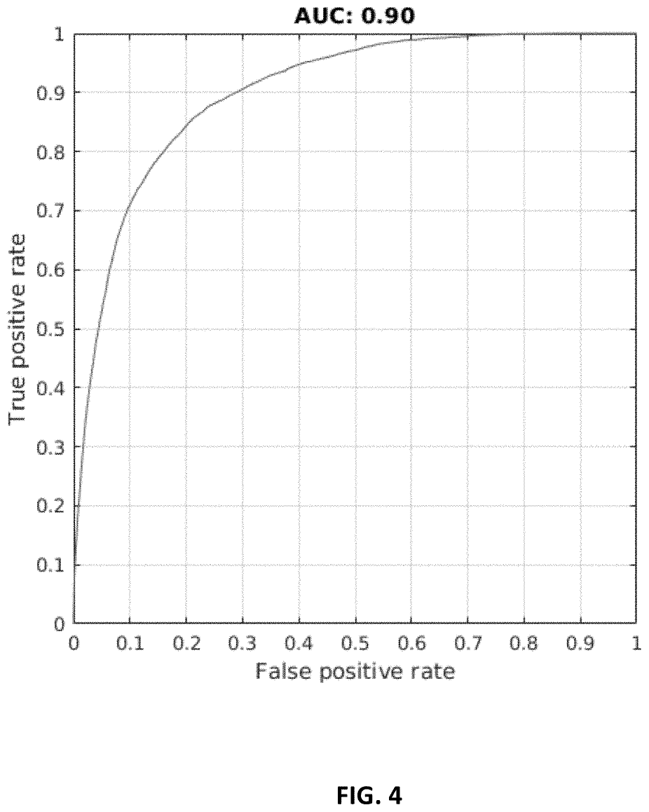

FIGS. 3A-3C show the results from three out of the five samples in the test set: a positive control, a negative control, and a mouse tumor with bound MBs. For each sample, the B-mode images of the nondestructive fundamental and harmonic datasets are shown alongside the “ground truth” (y) and the predicted (ŷ) MB locations. In FIG. 3A, six microvessel channels containing MB s were visible in the nonlinear harmonic mode but not in the fundamental mode. The network detected the presence of MB s in five out of the six microvessels. However, the network failed to detect the microvessel with an anomalously bright appearance in the fundamental mode image. In FIG. 3B, USMI images were obtained in a mouse tumor prior to MB injection, i.e., no MBs were present. The network predicted zero pixels with a MB probability of greater than 0.5, indicating accurate non-detection. In FIG. 3C, images were acquired in a tumor 7 min. post-injection of targeted MBs. The network prediction showed close correspondence to the destruction-subtraction image, with MBs detected inside the tumor located in the lower half of the image, and no MBs detected in the surrounding gel or non-tumor tissue in the upper half. FIG. 4 plots the ROC curve of the network prediction in FIG. 3C. Across the four images containing MBs, the network achieved a mean AUC=0.91 and DC=0.56 relative to the destructive subtraction images.

These results indicate that the neural network was able to distinguish MB signal from background tissue and noise using only the nondestructive dual-frequency channel data. Moreover, the quality of the results was comparable to that acquired using destruction-subtraction SLSC imaging, with accurate MB detection in the positive and negative controls as well as in the tumors. This shows that, through repetitive training, the network learned to detect characteristic frequency-dependent channel signal response of the MBs present in the nondestructive signals.

In another exemplary embodiment, the same NN was modified to accept different combinations of input data and trained with the same protocol. Nine separate configurations were compared: 1) Fundamental frequency 10 MHz only, denoted X_{f}; 2) Fundamental frequency 5 MHz (positive polarity) only, denoted Xₚ; 3) Sum of positive and negative polarity 5 MHz, denoted Xₕ; 4, 5, 6) Concatenation of Xₚ and Xₕ in channel data form, channel sum form, and detected envelope form, denoted Xₚₕ, Xₚₕ^{sum}, and Xₚₕ^{env}, respectively; 7, 8, 9) Concatenation of X_{f} and Xₕ in channel data form, channel sum form, and detected envelope form, denoted X_{fh}, X_{fh}^{sum}, and X_{fh}^{env}, respectively. For each of the nine configurations, the networks were trained across a range of learning rates ranging from 10⁻⁵ to 10⁻¹ by employing Bayesian hyperparameter optimization over 100 iterations.

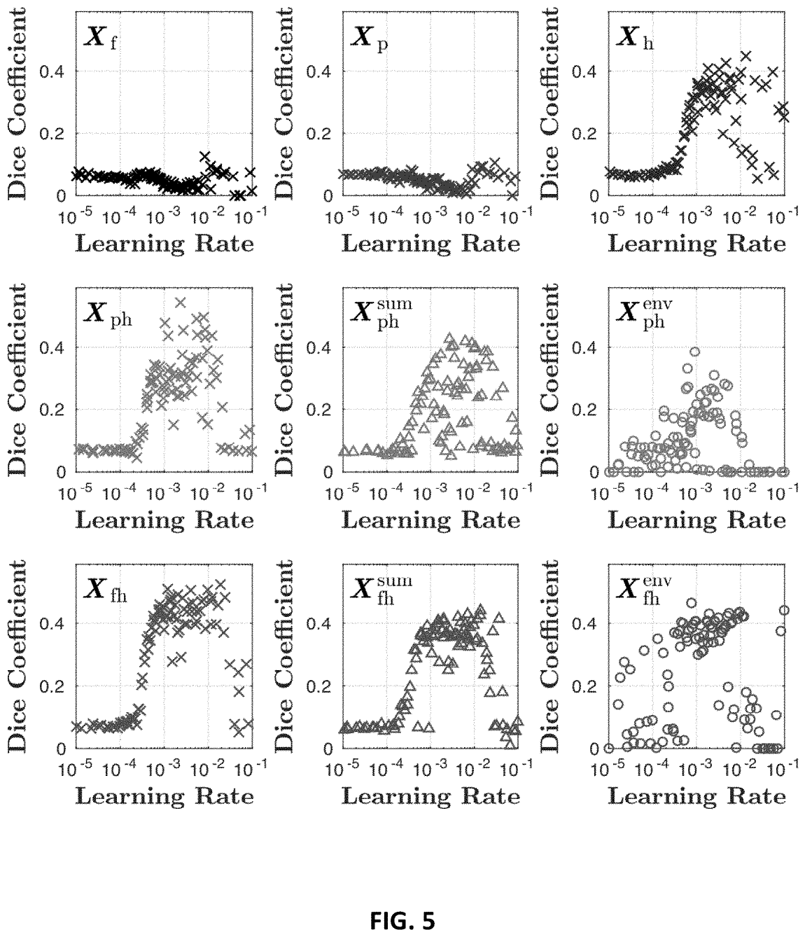

FIG. 5 shows the Dice coefficients as a function of learning rate for each of the 9 different configurations of input data. The networks which used fundamental frequency channel signal inputs only (X_{f}, Xₚ) failed to learn, giving low Dice coefficients. The network using the harmonic channel signals alone as input (Xₕ) was able to learn but performed suboptimally as compared to other input types. Providing Xₚ and Xₕ together as input to the network increased the Dice coefficient by the greatest amount when using channel data (Xₚₕ), and the least when using the envelope detected image (Xₚₕ^{env}). However, learning was inconsistent, with the same learning rates leading to a wide range of results. Learning was particularly consistent and successful when providing X_{f} and Xₕ together as input to the network. In particular, using channel data (X_{fh}) was more effective than using the channel sum (X_{fh}^{sum}) or the envelope detected images (X_{fh}^{sum}) as input. Overall, the highest Dice coefficients were achieved consistently when using X_{fh} as input.

The manually segmented destruction-subtraction SLSC images were treated as ground truth in this example. Although destruction-subtraction is currently considered the gold standard for MB confirmation, even these images contained significant amounts of noise, leading to a potential mislabeling of pixels. For instance, it was unclear in FIG. 3A whether the undetected microvessel contained MBs or an air bubble due to a lack of perfusion, leading to its distinct appearance in the fundamental image. In the case of the in vivo examples (e.g., FIG. 3C), the locations of the tumor vasculature (and thus the MB positions) were not known a priori, making the destruction-subtraction the best available estimate for their true positions. Despite the potential for mislabeling, neural networks have been proven to be capable of learning using noisy labels, motivating the continued use of destruction-subtraction imaging as ground truth.

An important consequence of these exemplary results is that MBs were detected nondestructively using the neural network beam-former, a critical step towards enabling safe and real-time USMI for the translation to clinical applications.

To summarize these examples, a novel neural-network-based beamformer is provided for the purpose of achieving safe and real-time USMI. The network was designed to utilize nondestructive channel data acquired at two distinct frequencies, and to produce a pixel-wise estimate of MB probability. The network was trained using a total of 20 USMI datasets acquired in a mouse model of hepatocellular carcinoma and in microvessel flow phantoms. The network was then evaluated on 5 distinct datasets: a positive control, negative control, and three previously unseen mouse tumors. Across the 5 datasets, the neural network achieved a mean AUC of 0.91 and DC of 0.56 compared to the destruction-subtraction images. These results demonstrate that a neural network can nondestructively distinguish MBs from background tissue and noise by exploiting characteristic differences in their fundamental and harmonic responses. The network was also found unable to learn when using only fundamental frequency data as input, was able to learn suboptimally when using only harmonic frequency data as input, and learned optimally when using both fundamental and harmonic data together. The nondestructive dual-frequency DNN beamformer enables safe and real-time USMI and can aid in the translation to clinical applications.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For example, the invention can be used any transmit pulse sequence, including diverging wave transmissions, focused transmissions, and coded excitations. The invention can be used with different combinations of ultrasonic frequencies and harmonics beyond the fundamental and second harmonics. Alternative preprocessing and post-processing can be performed besides channel downsampling and manual segmentation. The same methodology applies to alternative contrast agents with similar frequency characteristics to microbubbles, such as “nanodroplets” or “nanobubbles”, or microbubbles that have been loaded with a therapeutic agent. The ground truth images for training the neural network can be obtained using any variety of contrast agent imaging, including but not limited to difference imaging, spatial coherence imaging, acoustic angiography, and acoustic radiation force-induced motion imaging techniques. More sophisticated neural network architectures than the one employed here could yield improved results. The invention can be used for volumetric imaging in conjunction with a translating arm, such as an automated breast volume scanner system, or using matrix array transducers.

All such variations are considered to be within the scope and spirit of the present invention as defined by the following claims and their legal equivalents.

What is claimed:

1) A method of nondestructively detecting targeted contrast agents in real-time, comprising using a neural network (NN) beamformer, wherein an input of said NN comprises ultrasound transducer channel data from a dual-frequency pulse-echo acquisition from a medium that may contain targeted contrast agents, wherein an output of said NN is an image of pixel-wise probability of said targeted contrast agent presence, wherein said NN nondestructively distinguishes said targeted contrast agent from tissue and noise by exploiting to characteristic differences in responses of said targeted contrast agent versus responses from said tissue and noise present in said channel data of said dual-frequencies, wherein said NN is trained to operate according to destructive-subtraction ultrasound molecular imaging datasets that are used as a ground truth.

2) The method according to claim 1, wherein said NN is configured to acquire interleaved fundamental and harmonic frequency channel data, wherein said fundamental frequency acquisition comprises one set of pulses at an imaging frequency, wherein said harmonic frequency acquisition comprises two sets of pulses at half of said imaging frequency, wherein said harmonic frequency comprises opposite polarities that are summed.

3) The method according to claim 1, wherein said NN is configured to acquire fundamental and harmonic frequency channel data, wherein said fundamental frequency acquisition comprises one set of pulses at half of an imaging frequency, wherein said harmonic frequency acquisition comprises a sum of said set of pulses at half of said imaging frequency with a second matching set of pulses at half of said imaging frequency with opposite polarities.

4) The method according to claim 1, wherein said NN is configured to acquire harmonic frequency channel data, wherein said harmonic frequency acquisition comprises two sets of pulses at half of said imaging frequency with opposite polarities that are summed.

5) The method according to claim 1, wherein said dual-frequency pulse-echo acquisitions are performed using a plane wave or diverging wave synthetic transmit aperture technique.

6) The method according to claim 1, wherein said channel data acquisition is comprised of the radiofrequency data acquired on all transducer elements.

7) The method according to claim 1, wherein said channel data acquisition is comprised of a downsampled form of the radiofrequency data acquired on all transducer elements.

8) The method according to claim 1, wherein said NN is trained to identify said contrast agents according to destructive-subtraction images that are used as said ground truth, wherein each said destructive-subtraction image is formed by acquiring a pre-destruction image, eliminating said contrast agents from an imaging field of view using destruction, and subtracting a post-destruction image from said pre-destruction image, wherein said pre-destruction and post-destruction images are each formed by averaging a group of said channel data acquisitions comprising up to 30 frames and subsequently beamforming.

9) The method according to claim 1, wherein said pre-destruction and post-destruction images are reconstructed using a beamforming method selected from the group consisting of delay-and-sum beamforming, and SLSC beamforming, wherein said destructive-subtraction images are further enhanced using manual segmentation and image post-processing to eliminate artifacts.

10) The method according to claim 1, wherein training of said NN comprises:
a) obtaining a pre-destruction dual-frequency channel data acquisition;
b) passing said dual-frequency channel data acquisition into the NN to estimate a map of pixel-wise probability of the presence of said contrast agent (ŷ);
c) applying a strong destructive pulse to eliminate contrast agents from an imaging field of view and forming a ground truth destructive-subtraction image (y); and
d) comparing said (ŷ) versus (y) using a loss function, and to update the parameters of the neural network to minimize the loss function during said training.
